(19) United States
(12) Patent Application Publication
(10) Pub. No.: US 2008/0188719 A1
(43) Pub. Date: Aug. 7, 2008

(54) Health Test on the Fly

(76) Inventor: Per Erik KEMLEN, Uppsala (SE)

Correspondence Address: BIRCH STEWART KOLASCH & BIRCH, PO BOX 747, FALLS CHURCH, VA, 22040-0747, US

(21) Appl. No.: 11/672,029

(22) Filed: Feb. 6, 2007

(51) Int. Cl. A61B 5/00

(52) U.S. Cl. 600/300

(57) ABSTRACT

A method for health testing comprises offering of at least one health test. The offering takes place in an area in which traveling persons spend a waiting time, whereby the offering is directed to the traveling persons. A health test order is received and ordered health tests are performed according to the health test order. Results of the ordered health tests are distributed to the traveling persons. A person having access to such an offer will experience that time, that otherwise would be non-productive and stressing, instead can be used to achieve a positive experience. The result for the person is that higher health awareness is achieved. Being informed about belonging to certain risk group can initiate a change in life style, and eventually reduced personal suffering. The present health test approach also presents large advantages to the society as a whole, such as reduced need for medical treatment due to early diagnosis and of a reduced load on medical centers.

### TECHNICAL FIELD

The present invention relates in general to health testing and in particular to the enabling of quick health testing.

### BACKGROUND

Different kinds of welfare-related diseases increase rapidly in connection with changing lifestyles allover the worlds. High consumption of fat and sugar as well as a hectic way of living lead to an increased frequency of diseases such as diabetes, heart diseases, high blood pressure etc. Many of these diseases are possible to at least partially prevent by relative simple changes in lifestyle. However, in order to make such changes, many people need some concrete indication on that they belong to a certain high-risk group. Such indications are easily obtained by different kinds of health tests.

Health tests have been improved dramatically in recent years. Tests of most important risk factors of important diseases are now available, which can be performed within a very short time, without very large need of laboratory equipment, and to a relatively inexpensive price. Nevertheless, very few people do perform such tests. One reason for the low testing rate could be that people in general are not aware of the existence of such tests.

Another reason for the low testing rate is probably connected to the availability of the tests. Health tests are typically performed at hospitals or other medical or health centers. In order to be tested, the person to be tested has to contact the medical center, and typically reserve a time for the testing. Most available time is during daytime, which means that the person to be tested has to be free from his/her ordinary work and has to travel to the medical center, which costs both time and money. In many cases, the resources of the medical centers are also very limited and the queuing time for achieving a time for testing may be very long indeed. All such factors make people reluctant to proceed in trying to perform health tests, even if they know that such tests may be important for allowing a future healthy life.

### SUMMARY

A general object of the present invention is to provide an increased accessibility to health tests. A further object of the present invention is to provide health test in a manner that do not interfere with the productive time of the person to be tested and which not involve additional associated costs, such as traveling costs. Yet a further object of the present invention is to provide health tests in a manner that gives a positive total experience even during the actual testing time.

The above objects are achieved by methods according to the enclosed patent claims. In general words, a method for health testing comprises offering of at least one health test. The offering takes place in an area in which traveling persons spend a waiting time, whereby the offering is directed to the traveling persons. A health test order is received and ordered health tests are performed according to the health test order. Results of the ordered health tests are distributed to the traveling persons.

One advantage with the present invention, experienced by the user, is that time, that otherwise would be non-productive, can be used to achieve the positive experience of getting a better control over the personal health, i.e. a higher health awareness is achieved. Being informed to belong to a certain risk group can initiate a change in life style, and eventually reduced personal suffering. Furthermore, no costs in addition to the costs of the actual test are caused. The present invention also presents advantages to the society in form of cost savings for reduced need for medical treatment due to early diagnosis and of a reduced load on medical centers.

### BRIEF DESCRIPTION OF THE DRAWINGS

The invention, together with further objects and advantages thereof; may best be understood by making reference to the following description taken together with the accompanying drawings, in which:

FIG. 1 is a flow diagram of steps of an embodiment of a method according to the present invention;

FIG. 2 is a schematic drawing of an embodiment of a typical area in which the present invention advantageously is provided;

FIGS. 3A-C are schematic illustrations of embodiments of health test offers;

FIG. 4 is a view of a device for entering a health test order; and

FIGS. 5A-D are illustrations of embodiments of screen layouts of the device of FIG. 4.

### DETAILED DESCRIPTION

Many people today spend a lot of their time traveling. The travels can be commuting between a home and a place of work or long distance travels, either for business purposes or for tourism. In all cases, there are always waiting times at different stages. Airplane or boat passengers have typically to be present at the airport for checking-in a certain time in advance. People changing from one train to another spend some waiting time at the train station. People traveling by bus or car often spend some time at road service areas. Common for such situations is that there are lots of people present that more or less against their own desires have to spend some waiting time in a certain limited area.

Waiting travelers typically spend such times having a drink or snacks, reading a newspaper or book, or visiting any shops that may be available at the site. Many such activities are for many people experienced as simply time-destructive and are therefore connected with a negative value, possibly increasing the overall stress experience. Such an opportunity may instead, according to the present invention, be turned into a generally positive experience by focusing on health. A psychologically well selected occasion may therefore increase the mental accessibility to health tests.

By offering easily accessible health test in areas in which traveling persons spend a waiting time a positive value can be achieved by the very act of undergoing a health test. The health tests should be short and possible to be performed and possibly also evaluated within the average time the persons have to spend in the area. Instead of wasting time on unnecessary activities, the time can instead be spent on a matter having the potential of influencing the entire life in a positive direction. The psychological obstacles for initiating a health test procedure are probably much less if no extra productive time has to be sacrificed.

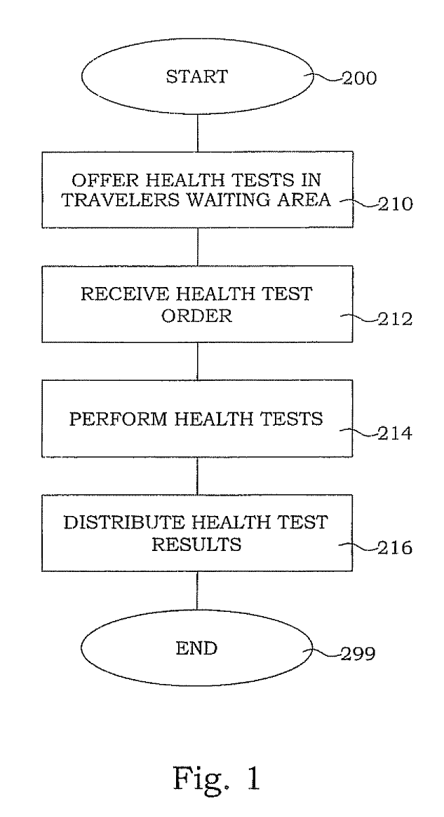

The basic principles can be illustrated by the flow diagram of FIG. 1. A method for health testing starts in step 200. In step 210, at least one health test is offered. The offering is performed in an area in which traveling persons spend a waiting time. The area in which traveling persons spend a waiting time can be a terminal, for e.g. trains, aircrafts, boats or buses. The area in which traveling persons spend a waiting time can also be a road service area. The health tests are therefore mainly directed to traveling persons being present in the area, using different communications, e.g. train, aircraft, boat, car or bus. In step 212, a health order is received. This health order can be provided orally, in written format or by electronic means. The ordered health tests are performed in step 214 according to said health test order. In step 216, results of the ordered health tests are distributed to the traveling persons. This distribution can also be performed by different means, e.g. orally, in written form directly in connection with the test, by mail, or by various electronic means. The procedure ends in step 299. The procedure may also be repeated one or more times.

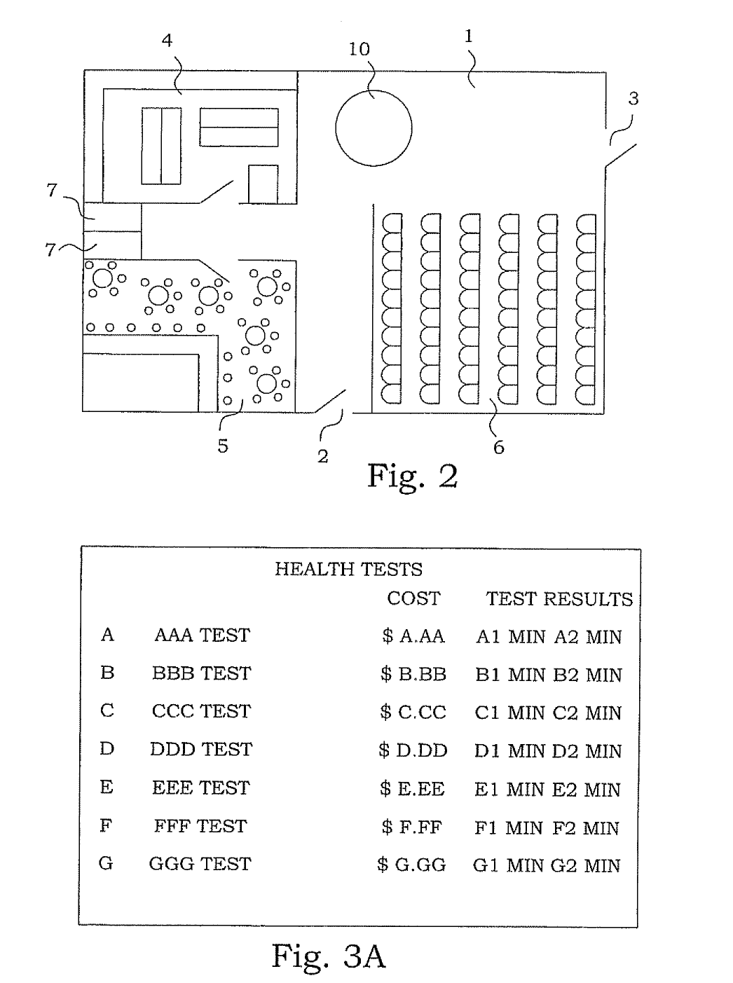

FIG. 2 is a schematic drawing of an area 1 in which traveling persons spend a waiting time. A traveler typically enters through an entrance 2, spends some time in the area 1 and exits through an exit 3. In the present example, a couple of service facilities are provided. A book store 4 sells magazines and books. A cafe or bar 5 provides drinks and food. A lounge 6 provides seats for anyone wanting to sit down. Toilets 7 are also provided. Travelers spending some time in such an area will typically after a while become restless, which increase the stress level. According to the present invention a health care booth 10 is provided in the area 1, offering instant health tests. Here, the travelers can, according to the above briefly described procedure, order a set of health tests. The health care booth 10 has preferably a round shape avoiding that queues are building up. If the tests may include activities where the traveler has to lie down or need some privacy, the health care booth may comprise separate compartments for such testing.

The offering of health tests is typically performed by advertising at posters in connection with the health care booth 10 and preferably also at other places within the area 1. The posters can in certain embodiments be electronically controlled posters. The offers may also be presented by flyers, e.g. at a desk of the health care booth. Advertisement for the offers may also additionally be distributed elsewhere, e.g. at travel agencies, check-in counters, insurance companies, travel companies, sports shops etc., in order to open the eyes for the possibilities offered by the present invention.

From the traveler's point of view, he enters the area 1 in which he has to spend a certain waiting time. The waiting time is most often not chosen by the person, but is forced on him by other circumstances. Besides typical time-killing activities such as food, drinks or literature, the present invention offers instant health tests. The traveler is readily informed about the possible tests, the target health aspect of the different tests and times connected with the tests, such as actual testing time and analysis time. Since many of the tests and even groups of tests are possible to perform during a time period that is smaller, and typically much smaller, than the expected waiting time, the testing appears to be easily available to the traveler. By having knowledge about testing times arid purposes of tests, an average person may easily compose a test set that is well suited for the person in question. If the traveler needs additional information, trained personnel is available for quick support even during the selection phase.

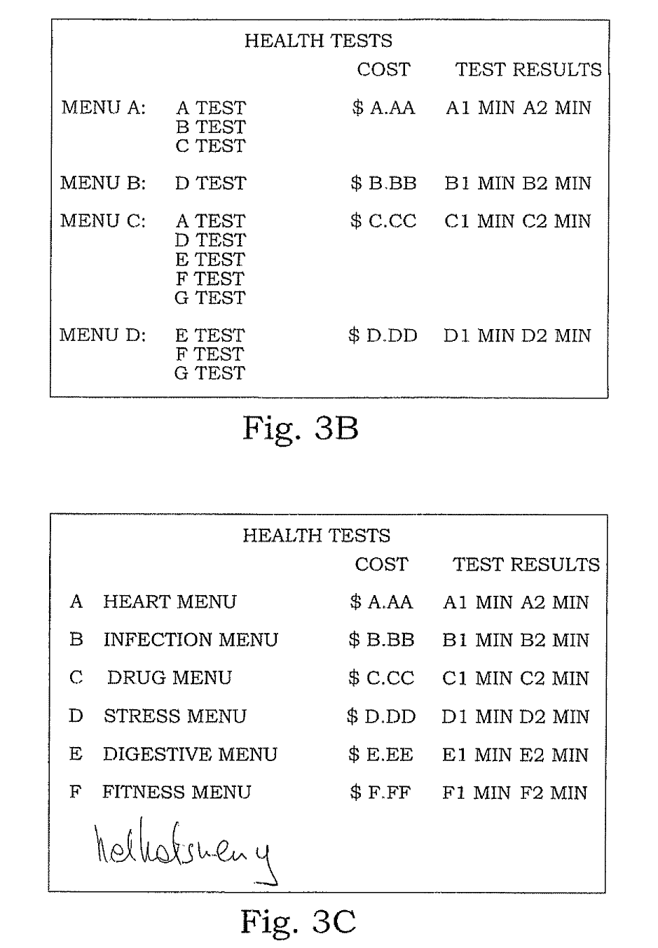

FIG. 3A illustrates one example of a test offer. A plurality of health tests are listed, which can be performed at the health care booth. In connection with each test, there is a price for the test as well as an average testing time. If there is an evaluation or processing time connected with the test, the time until a result is provided may be considerably longer than the actual testing time. In such cases, an average time to test results may also be declared. Non-exclusive examples of health test that may be offered and performed by the present invention are blood pressure tests, blood sugar tests, blood fat tests, blood sedimentation rate tests, c-reactive protein tests (CRP), blood group tests, pulse tests, infection tests, virus tests, bacterial tests, gravidity tests, lung capacity tests, lung function tests, drug tests, alcohol tests, heart frequency tests, oxygen transport tests, determination of weight, determination of fat percentage, determination of body mass index (BMI), body water, muscle mass, menopause tests, fertility tests, cancer tests, kidney function tests, gastrointestinal bleeding tests, reaction tests, vision tests, color-blindness tests and hearing tests. Tests are today available, which are easy and fast to perform. An option would also be to have test kits for sale at the health care booth 10. Most results are also available within a couple of minutes and can be presented orally, in written form or electronically. This makes it possible to complete a set of health tests within a reasonable time for a traveler.

In many cases, there are advantages by combining a set of tests at the same time. The tests can then give complementary information in cases where a single test may be ambiguous. However, other tests may instead be more or less alternatives for measuring the same aspects. Furthermore, if e.g. a test requires a blood sample, it won't be very difficult to perform also other tests requiring a blood sample. In such cases, a better price may be offered for certain combination of tests. For instance, blood pressure and pulse tests are easily performed simultaneously. FIG. 3B illustrates an offer for test packages or test menus. Each health test menu comprises a predetermined set of at least one health test. The packages are preferably composed to be either complementary in different medical senses or easy to perform at the same time. The health tests menus may then assist the traveler to select an appropriate set of health tests.

Different health tests are focusing on different health aspect. As illustrated in FIG. 3C, it could be particularly advantageous if offered health test menus are characterized by a specific health aspect. In FIG. 3C, a first menu is directed to cardiovascular diseases, e.g. different heart-related diseases, a second menu is directed to infections, a third to drug-related issues, a fourth to stress-related diseases, a fifth to digestive problems, a sixth to fitness etc. Other examples of health aspects can be endocrine diseases, including diabetes, lung diseases, cancer, fertility/infertility, kidney function, audible defects, reaction, or sight defects. Such information assists the traveler in selecting a set of health tests that is appropriate for the intentions of the traveler. For travelers having plenty of time to spend, a “total” menu, including all available test, can also be offered.

Anyone skilled in the art realizes that combinations of the offers illustrated in FIGS. 3A-C also may be utilized.

When a traveler has decided which health test or set of health tests he/she wants to have, a test order is given to the staff of the health care booth. This order can be an oral order. The staff will then serve the customer accordingly. However, preferably, the order is provided in a written form as a filled-in paper form. In this form, any necessary agreements may be included, e.g. secrecy agreements. The staff receives the filled-in form and serves the customer accordingly.

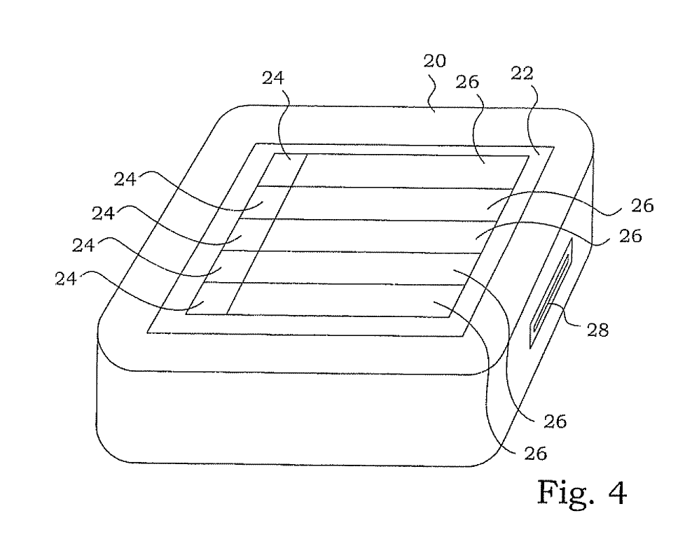

The reception of the health test order may also include electronic receiving of the health test order. In such a case, necessary information can easily be provided e.g. by use of a touch screen. FIG. 4 illustrates schematically a device 20 for entering a health test order. The device 20 comprises a touch screen 22. The touch screen 22 is controlled to visualize a number of fields 24, 26, which may comprise information of different kinds. Some fields, in the present embodiment fields 24, are touch sensitive fields. When a customer touches such areas, the device 20 interprets the touch as a data entry. By presenting a number of screens, data concerning the health test order can easily be collected. The device 20 preferably also comprises a credit card reader 28, whereby debiting issues also can be handled in an easy way.

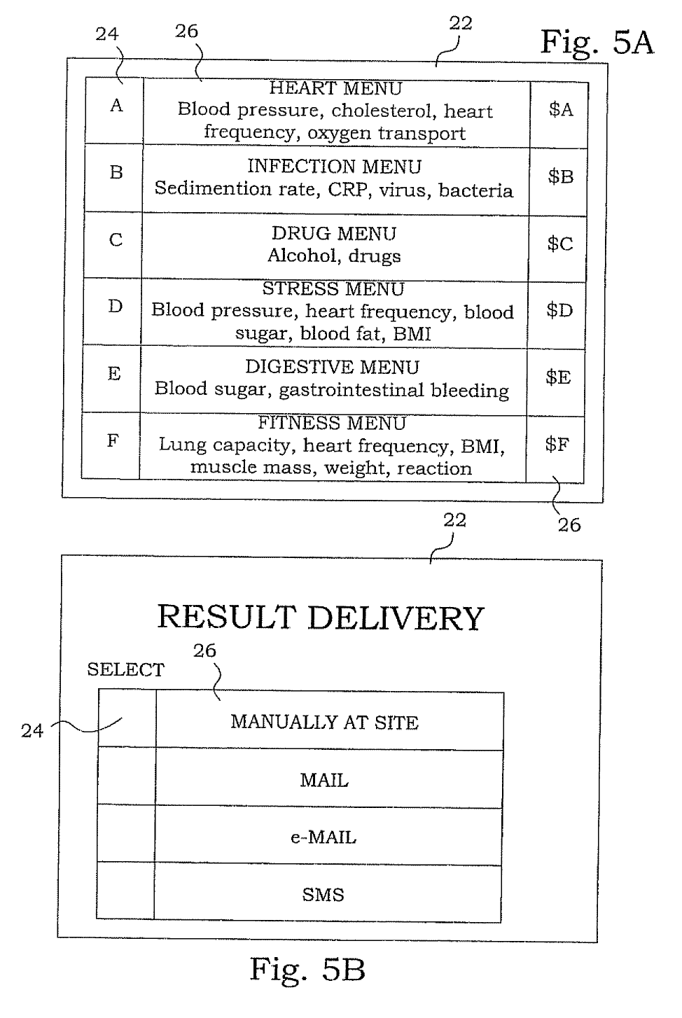

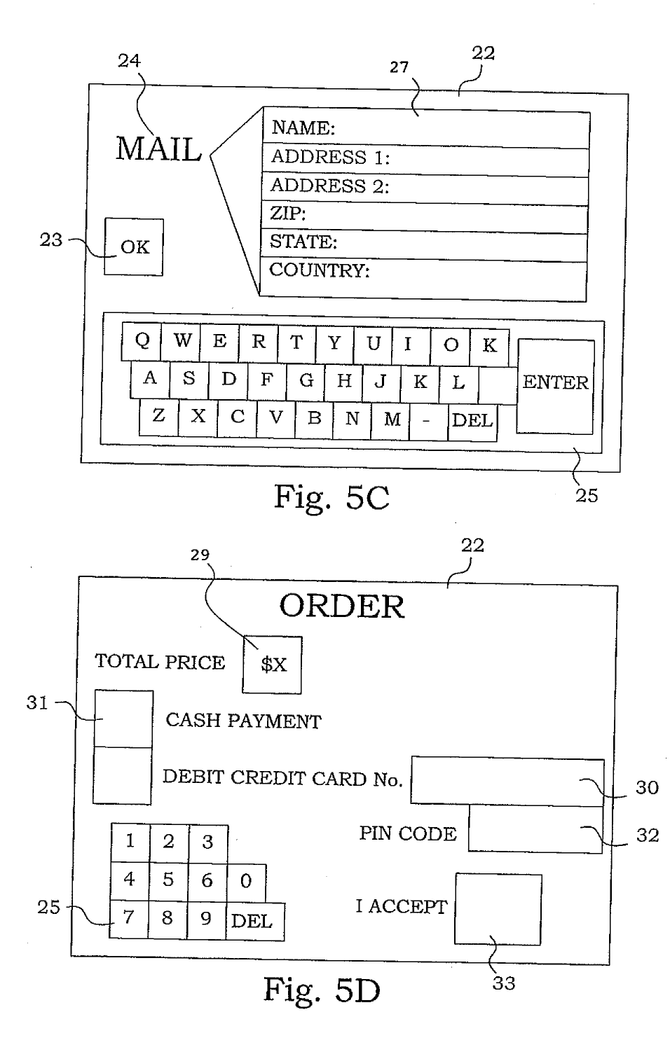

FIG. 5A illustrates an embodiment of a screen layout for entering an electronic order. A number of test menus are presented in information fields 26 as well as the price for the different tests. Select fields 24 are provided, which are sensitive for touching, and by which a certain test menu can be selected. E.g. by touching the letter B, a set of health tests suitable for, stress-related issues is selected.

FIG. 5B illustrates a next screen layout. In this layout, the customer can decide how the results of the tests should be delivered to the customer. In a typical case, the results are delivered in written form directly at the desk. If tests are selected, that cannot be accounted for within a reasonable time, other result distributing has to be used. This could e.g. be the case where the actual tests are performed rapidly, but the analysis takes some time. The analysis may even be longer than the expected customer waiting time. The customer may therefore choose to perform the test, but having the analysis result distributed at a later occasion. Also, if the customer of some reasons does not wish to know the results until a later occasion, other distributing options may be selected. For example, someone traveling on vacations may save the test results until he/she returns home in order not to influence his/her possibilities to relax on the vacation. However, other persons may request an immediate answer in order to be able to relax. The choice is therefore made from one customer to another. By touching the select field 24 marked by “mail”, a delivery of the result by ordinary postal services is selected.

In such a case, an address to the customer is required. A screen layout as presented in FIG. 5C may then be of use. Here, a number of name and address fields 27 are to be filled in. A touch screen keyboard 25 is also presented at the touch screen display. The customer touches a field to be filled in to activate that particular field, touches the letters of the keyboard 25 to enter information in the field. When all necessary information is available, the confirmation field 23 is touched.

Payment instructions may also be a part of the ordering. A screen layout according to FIG. 5D may then be used. A total price for the selected tests is presented in a price field 29. If a credit card is entered into the order device, a card number 30 can be retrieved automatically. Otherwise, the customer has to manually enter the credit card number by using the number touch screen keyboard 25 or select a field 31 that indicates that the tests are to be paid by cash. A personal identification code 32, a so-called PIN code has to be entered to verify that the customer is the authorized owner of the credit card. When all data is provided, a field 33 is touched in order to accept the payment terms. Other identification means, e.g. based on biometric identifying could also easily be utilized in the present invention. In such a way, a fingerprint or an eye scanning could be used for authorising payment as well as for identifying the person to be tested.

When the health test order is received, the health tests are performed accordingly. Tests requiring moderate impact on the personal integrity may be performed directly at the counter, if the customer so allows, e.g. acquiring a blood sample. For customers so requesting and for more extensive testing, isolated testing booths are preferably provided, e.g. provided with a couch. Booths having special equipment for certain tests may also be provided.

The tests are performed according to a respective procedure. The different particulars of the tests are known in prior art and are not of very high importance for the present inventive idea. The actual tests are therefore not described in any more detail.

When the results are available, distribution of results can, as discussed above, be performed in different manners. The results may e.g. be distributed directly in the area in which traveling persons spend a waiting time. Preferably, the results are provided in written form. The results may also be distributed by postal services to an address given by the customer. Another way of distributing results is to utilize different kinds of telecommunication services. Nonexclusive examples are e-mail, SMS (Short Message Service), MMS (Multimedia Message Service) and telefax, Test results may also be distributed to another health booth, linked to the one making the tests. The traveler may thereby pick up the results at a later stage of a scheduled travel route.

Distribution of results is preferably performed with some kind of authorization, authentication, privacy or encryption functionality in order to prohibit unauthorized persons to acquire information or to prohibit false information to be distributed.

When the results are provided, comparative figures of normal ranges of the distributed results are preferably provided together with the test results. This enables the customer to interpret the test results in terms of “normal” or “out of normal”. Preferably, the comparative figures are accompanied with further explanations, such as if the test results indicates an acute health hazard, if further tests are to recommend soon or if any deviations from normal are of marginal nature.

In a preferred embodiment, the tests are distributed together with a health diagnosis based on said distributed results. Such a health diagnosis may be automated in certain cases where the diagnosis is non-complex. In more complex cases, the health diagnosis is preferably performed manually by anyone skilled in the profession of interpreting such test results, e.g. a physician a well-educated nurse or laboratory assistant. Recommendations for treatment and/or life style changes may accompany the test results.

The primary target group of the present invention is travelers. However, anyone skilled in the art realizes that also other groups of people may use such health testing. If the health test booth for instance is provided at an airport, it might be interesting for personnel of e.g. air flight companies, in particular flying personnel, service facility personnel etc. to make health tests. It might even be interesting to give certain groups of people, often visiting the area, different discounts possibilities.

The embodiments described above are to be understood as a few illustrative examples of the present inventions. It will be understood by those skilled in the art that various modifications, combinations and changes may be made to the embodiments without departing from the scope of the present invention. In particular, different part solutions in the different embodiments can be combined in other configurations, where technically possible. The scope of the present invention is, however, defined by the appended claims.

1. A method for health testing, comprising the steps of:
offering at least one health test;
said step of offering being performed in an airport terminal in which traveling persons to be traveling by aircraft spend a waiting time waiting for an aircraft flight;
whereby said offering step being directed to said traveling persons;
receiving a health test order;
performing ordered health tests according to said health test order; and
distributing results of said ordered health tests to said traveling persons.

2-4. (canceled)

5. The method according to claim 1, wherein said step of distributing is performed in said airport terminal.

6. The method according to claim 1, wherein said step of distributing is performed by postal services.

7. The method according to claim 1, wherein said step of distributing is performed by telecommunication services.

8. The method according to claim 7, wherein said step of distributing is performed by one of:
e-mail;
SMS;
MMS; and
telefax.

9. The method according to claim 1, wherein said step of distributing is performed at a different airport terminal.

10. The method according to claim 1, wherein said step of offering comprises a plurality of health tests.

11. The method according to claim 10, wherein said step of offering comprises offering of a number of health test menus, where each health test menu comprises a predetermined set of at least one of said plurality of health tests.

12. The method according to claim 11, wherein each of said health test menus are directed to a specified health aspect.

13. The method according to claim 12, wherein at least one of said specified health aspects is selected among:
cardiovascular diseases;
heart diseases;
stress;
infections;
drugs;
digestive problems;
endocrine diseases;
diabetes;
lung diseases;
cancer;
fertility /infertility;
kidney function;
fitness;
audible defects;
reaction and
sight defects.

14. The method according to claim 1, wherein said step of distributing further comprises distributing of comparative figures of normal ranges of said distributed results.

15. The method according to claim 1, wherein said step of distributing further comprises distributing of a health diagnosis based on said distributed results.

16. The method according to claim 1, wherein said step of receiving comprises receiving a filled-in paper form,

17. The method according to claim 1, wherein said step of receiving comprises electronic receiving of said health test order.

18. The method according to claim 17, wherein said step of receiving is performed via a touch screen.

19. The method according to claim 1, wherein at least on of said at least one health test is selected from the list of
blood pressure tests;
blood sugar tests;
blood fat tests;
blood sedimentation rate tests;
c-reactive protein tests;
blood group tests;
pulse tests;
infection tests;
virus tests;
bacterial tests;
gravidity tests;
lung capacity tests;
lung function tests;
drug tests;
alcohol tests;
heart frequency tests;
oxygen transport tests;
determination of weight;
determination of fat percentage;
determination of body mass index;
determination of body water;
determination of muscle mass;
menopause tests;
fertility tests;
cancer tests;
kidney function tests;
gastrointestinal bleeding tests;
reaction tests;
vision tests;
color-blindness tests; and
hearing tests.
